(19) United States
(12) Patent Application Publication
(10) Pub. No.: US 2020/0060864 A1
(43) Pub. Date: Feb. 27, 2020

(54) MENSTRUAL CUP

(71) Applicant: Ramon Vendrell Vila, Balsareny, Barcelona (ES)

(72) Inventor: Ramón Font Caselles, Manresa, Barcelona (ES)

(21) Appl. No.: 16/305,457

(22) PCT Filed: May 31, 2017
(86) PCT No.: PCT/ES2017/000063

§ 371 Date: Nov. 29, 2018

(30) Foreign Application Priority Data
Jun. 1, 2016 (ES) U-210600375

(51) Int. Cl. A61F 5/455; A61F 5/44

(52) U.S. Cl. CPC A61F 5/4553; A61F 5/4407; A61F 5/4408

(57) ABSTRACT

A menstrual cup comprising a hollow body including an upper opening defined by a rounded peripheral rim; a hollow cylindrical portion of smaller diameter than the peripheral rim, the hollow cylindrical portion being connected to a lower hollow portion of smaller diameter and decreasing cross-section, which is closed at the base thereof and extends into a grip extension. The lower portion and the grip extension are provided with peripheral ribs. The cylindrical portion comprises small holes through which air can pass into the body, preventing the creation of a vacuum therein. The cross-section of the aforementioned holes decreases towards the exterior of the hollow body. The grip extension of the cup includes a hollow cylindrical segment that terminates internally in a hollow head provided with an end opening.

### FIELD OF THE INVENTION

This invention is applicable to the field of intimate feminine hygiene and more particularly, to a menstrual cup applicable for collecting menstrual discharge.

### STATE OF THE ART

The first menstrual cups were manufactured in plastic around 1930, and they have been the object of many improvements, both regarding the type of materials used and their design, with the aim of improving their performance and comfort.

The utility model ES1073037U describes a reusable hypoallergenic menstrual cup comprising a body in the shape of a cup, the upper edge thereof comprising a rounded edge protruding from the outer cup ending in an area shaped as a circular ring, which continues as an inverted tapered shape that terminates in a pendulum in the shape of a solid sphere, which is joined to the cup by means of a cylindrical neck, also solid, that comprises solid rings for outer reinforcement on both ends.

Menstrual cups of this kind have some drawbacks when used, particularly during the removal thereof, since due to their aforementioned shape in the form of a cup and due to comprising a flexible material, in some circumstances they can exert a suction effect.

This suction effect requires applying a determined pulling force on the base of the cup; in order to do so, the aforementioned background comprises on the base thereof a pendulum in the shape of a solid sphere joined to the cup by means of a cylindrical neck, also solid, with solid reinforcement rings. The solid nature of the aforementioned pendulum and the reinforcement rings causes the base of the cup to have a higher consistency than the rest of the cup, which may cause discomfort to the user while using the cup.

It should also be highlighted that although there are known documents in the state of the art that divulge alternative solutions, the applicant is unaware of the existence of menstrual cups that comprise technical, structural and constituent characteristics similar to those of the menstrual cup that is the invention.

### SUMMARY OF THE INVENTION

The menstrual cup that is the invention is of the kind mentioned above, and it comprises a hollow body, including from top to bottom: an upper opening defined by a rounded peripheral rim; a hollow cylindrical portion of smaller diameter than the peripheral rim, said hollow cylindrical portion being connected to a lower hollow portion of smaller diameter and decreasing cross-section, which is closed at the base thereof and extends into a grip extension; with the lower portion and the grip extension provided with peripheral ribs to improve the grip thereof.

This menstrual cup presents a series of structural particularities designed to address the problem detailed above, preventing the generation of a vacuum therein that may make it more difficult to remove. Said cup defines in the lower gripping area a configuration with a lower consistency than that of the aforementioned background, which makes it possible to reduce the risks of discomfort to the user while using the menstrual cup.

Another object of the invention is to provide the peripheral ring defined in the upper opening of the cup with a configuration at the base thereof that makes it easier to remove the menstrual cup after being used.

In order to achieve these objectives, the menstrual cup comprises in the cylindrical portion thereof a series of holes through which air can pass into the interior space of the hollow body, preventing the creation of a vacuum therein.

Said holes have a cross-section, preferably conical, that decreases towards the exterior of the hollow body, with the aim of allowing the passage of air and preventing the discharge collected by the menstrual cup from exiting.

The grip extension of the cup comprises a hollow cylindrical segment that terminates internally in a hollow head provided with an end opening; with said grip extension providing better flexibility and comfort.

Since the grip extension is hollow, said extension flattens and increases the gripping surface thereof when it is pressed in order to remove the cup.

Another characteristic of the invention is that the peripheral rim comprises an upper portion with a circular cross-section that is connected on the exterior thereof with the cylindrical portion by means of a peripheral ramp with a square generatrix; said peripheral ramp facilitating the removal of the menstrual cup after being used.

### BRIEF DESCRIPTION OF THE FIGURES

To complement the description being made, and to make it easier to understand the characteristics of the invention, this descriptive report is accompanied by a set of drawings in which, for illustrative purposes only, the following has been represented:

FIG. 1 shows an upper view of an example embodiment of the menstrual cup, according to the invention;

FIG. 2 shows an upper view of the menstrual cup in the previous figure with a vertical median plane cross-section; and

FIG. 3 shows an upper plan view of the menstrual cup in the previous figures.

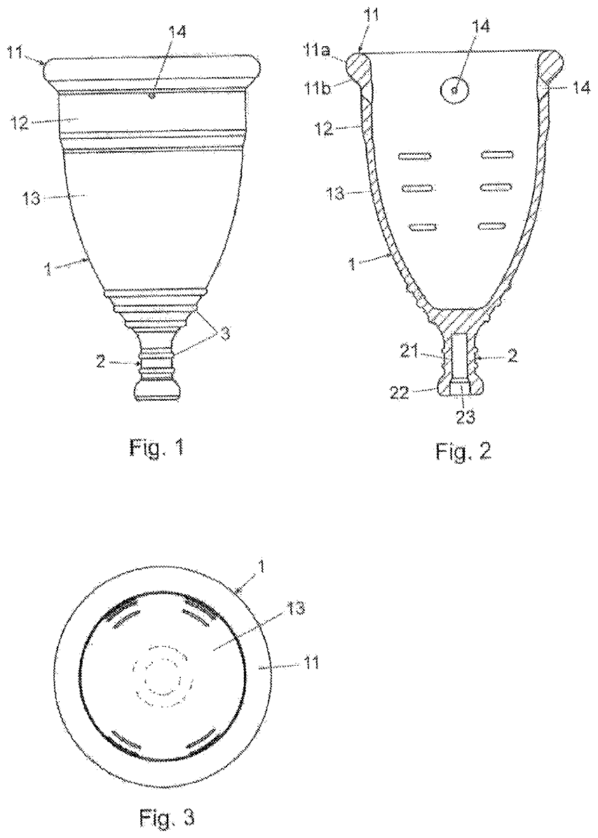

### PREFERRED EMBODIMENT OF THE INVENTION

As the figures attached show, the menstrual cup comprises a hollow body (1) comprising a grip extension (2) and peripheral ribs (3) at the base thereof in order to improve the grip thereof; with the entirety of the cup being formed of medical silicon.

The hollow body (1) is a body of revolution, comprising: an upper opening defined by a rounded peripheral rim (11); a hollow cylindrical portion (12) of smaller diameter than the peripheral rim (11), said hollow cylindrical portion being connected to a lower hollow portion (13) of smaller diameter and decreasing cross-section, which is closed at the base thereof.

The cylindrical portion (12) comprises small holes (14) through which air can pass into the body (1), preventing the creation of a vacuum therein. As the cross-section of FIG. 2 shows, said holes (14) have a cross-section, preferably conical, that decreases towards the exterior of the hollow body (1), with the aim of allowing the passage of air and preventing the discharge collected by the menstrual cup from exiting.

The peripheral rim (11) comprises an upper portion (11a) with a circular cross-section that is connected on the exterior thereof with the cylindrical portion (12) by means of a peripheral ramp (11b) with a square generatrix; said peripheral ramp (11b) facilitating a gentle removal of the menstrual cup.

The grip extension (2) of the cup comprises a hollow cylindrical segment (21) that terminates internally in a hollow head (22) provided with an end opening (23).

Having described sufficiently the nature of the invention, and the example of a preferred embodiment, it can be stated for the relevant effects that the materials, shape, size and arrangement of the elements described may be modified, provided this does not mean an alteration in the essential characteristics of the invention for which the claims are set out below and as allowed.

1. A menstrual cup comprising:
a hollow body (1) including from top to bottom: an upper opening defined by a rounded peripheral rim (11); a hollow cylindrical portion (12) of smaller diameter than the peripheral rim, said hollow cylindrical portion being connected to a lower hollow portion (13) of smaller diameter and decreasing cross-section, which is closed at the base thereof and extends into a grip extension (2); wherein the lower portion (13) and the grip extension (2) are provided with peripheral ribs (3) to improve the grip thereof;
wherein the cylindrical portion (12) comprises an inner surface and an outer surface, and wherein said cylindrical portion (12) comprises a plurality of small holes (14) extending from said inner surface to said outer surface through which air can pass into the hollow body (1), preventing the creation of a vacuum therein; wherein the holes (14) have a cross-section that decreases in diameter from said inner surface towards the outer surface of the hollow body (1); and further, wherein the grip extension (2) of the cup includes a hollow cylindrical segment (21) that terminates internally in a hollow head (22) provided with an end opening (23).

2. The menstrual cup according to claim 1, characterised in that the peripheral rim (11) comprises an upper portion (11a) with a circular cross-section that is connected on the exterior thereof with the cylindrical portion (12) by means of a peripheral ramp (11b) having a generally linear outer surface.
